(19) United States
(12) Patent Application Publication
(10) Pub. No.: US 2023/0398535 A1
(43) Pub. Date: Dec. 14, 2023

(54) SAMPLE ANALYSIS CARTRIDGE AND SYSTEM

(71) Applicant: TTP PLC., Royston, Hertfordshire (GB)

(72) Inventors: Piers Sebastian Harding, Royston, Hertfordshire (GB); Gary Keith Jepps, Royston, Hertfordshire (GB)

(21) Appl. No.: 18/033,089

(22) PCT Filed: Oct. 20, 2021
(86) PCT No.: PCT/GB2021/052718

§ 371 Date: Apr. 21, 2023

(30) Foreign Application Priority Data
Oct. 21, 2020 (GB) 2016715.1

(51) Int. Cl. B01L 3/00; B01L 7/00

(52) U.S. Cl. CPC B01L 3/502715; B01L 7/52; B01L 2400/0487; B01L 2200/0689; B01L 2300/123; B01L 2300/0654; B01L 2300/1805; B01L 2200/04

(57) ABSTRACT

There are provided sample analysis cartridges, and methods and systems using such sample analysis cartridges, wherein the sample analysis cartridges comprise a reaction chamber configured to receive a sample; a film defining at least a portion of a face of the reaction chamber; an inlet port fluidly connected to an inlet of the reaction chamber and an outlet port fluidly connected to an outlet of the reaction chamber; and an air reservoir fluidly connected to the reaction chamber; the inlet port, outlet port and air reservoir arranged such that, as the reaction chamber is filled with fluid through the inlet port, gas within the reaction chamber may escape the cartridge through the outlet port whilst gas within the air reservoir is retained; the inlet port, outlet port and air reservoir being further arranged such that such that, if the inlet and outlet ports are sealed, gas within the air reservoir applies pressure on the contents of the reaction chamber.

### FIELD

The present invention relates to cartridges and systems for preparing and analysing samples and methods of preparing and analysing samples using such devices. The invention provides fast and accurate preparation and analysis of samples, and a quick and convenient disposal of samples after use.

### BACKGROUND

In the field of diagnostics there has been a growing need to provide sample preparation and analysis devices that can be used in the analysis of a sample from a patient. In particular there has been a growing need for ‘point-of-care’ medical diagnostic devices that enable a sample to be analysed at the location of a patient to ensure rapid analysis and to improve overall care for the patient.

One analytical approach which is desired to be implemented in such a point-of-care device is polymerase chain reaction (PCR). PCR is a technique in which small samples of segments of DNA and RNA are amplified or copied to provide larger quantities of the sample to study in detail. During this process a purified eluate containing the DNA or RNA in question is thermal cycled to amplify and detect the DNA or RNA in question.

The design of systems for use in PCR and similar processes face a number of challenges. Firstly, it is desirable for the temperature of a fluid sample to be accurately controlled and for the distribution of temperature to be uniform throughout a sample during thermal cycling. It is also desirable to be able to vary the fluid temperature at a high rate. Preferably outgassing—the formation of bubbles as gas comes out of solution during heating—is prevented during thermal cycling as such bubbles can impact subsequent analysis steps (e.g. optical analysis). Furthermore, it is critical that leakage of the sample (e.g. the amplicon during PCR) is prevented during thermal cycling, during analysis, or after use. Any release or escape of a sample can cause significant contamination of a laboratory and equipment, rendering subsequent test results invalid. Finally, systems preferably are capable of accurately and precisely analysing and detecting the contents of a sample following thermal cycling.

Certain existing designs are capable of addressing one or more of these challenges at the expense of others. For example, some existing systems are capable of performing thermal cycling quickly but as offer low detection sensitivity.

Others provide acceptable thermal and optical performance but have the potential to leak and contaminate the work environment.

There is furthermore a need to provide a sample analysis systems and cartridges which are small in size and weight, as well as being easy to manufacture and of low cost.

There is therefore a need for improved sample preparation and analysis systems that overcome at least some of—and preferably all of—the challenges and problems identified above.

### SUMMARY OF INVENTION

In accordance with an aspect of the invention there is provided a sample analysis cartridge, comprising: a reaction chamber configured to receive a sample; a film defining at least a portion of a face of the reaction chamber; an inlet port fluidly connected to an inlet of the reaction chamber and an outlet port fluidly connected to an outlet of the reaction chamber; and an air reservoir fluidly connected to the reaction chamber; the inlet port, outlet port and air reservoir arranged such that, as the reaction chamber is filled with fluid through the inlet port, gas within the reaction chamber may escape the cartridge through the outlet port whilst gas within the air reservoir is retained; the inlet port, outlet port and air reservoir being further arranged such that such that, if the inlet and outlet ports are sealed, gas within the air reservoir applies pressure on the contents of the reaction chamber. Cartridges in accordance with the invention provide means to pressurise fluid in the reaction chamber during thermal cycling or other sample preparation processes. The reaction chamber of the cartridge may be filled with a fluid such as a liquid sample via the inlet port. During this filling process gas, typically in the form of air, within the reaction chamber may exit from the cartridge via the outlet port, whereas gas is retained or stored within the air reservoir. If the inlet and outlet ports are subsequently sealed this stored or trapped gas (e.g. air) within the air reservoir exerts pressure on the fluid (e.g. a sample) within the reaction chamber. Indeed, it will be appreciated that the gas within the air reservoir acts as an energy store or “air-spring”, continuing to apply pressure to the fluid within the reaction chamber as the temperature of the cartridge and the reaction chamber is varied—e.g. during thermal cycling.

Pressurising the fluid in this manner acts to prevent outgassing, in which gas might otherwise come out of solution during heating. Thus the accuracy and sensitivity of optical analysis performed on a sample within the reaction chamber after heating and/or thermal cycling may be increased.

The film will be understood to be a thin, flexible sheet of material. Such flexible films may include a variety of materials including membranes and foils. The materials of these films may comprise metals (e.g. Aluminium) or polymers (e.g. polypropelene). Particularly suitable materials of products suitable use as a film in the invention include: AB-1720 Easy Pierce 20 μm Heat Sealing Foil produced by ThermoFisher Scientific®; and 4ti-0539 Aluminium Foil produced by 4Titude® Ltd. These product examples are of opaque laminate foils.

However, clear and/or colourless films produced by the same manufacturers are equally suitable for use in the invention.

The reaction chamber may be heated and cooled through the flexible film—e.g. by placing the cartridge against a thermal surface configured to heat and/or cool the contents of the reaction chamber. For instance the thermal surface may be a surface of heating blocks within a thermocycler, and/or the surface of a heater (e.g. a resistive, thin film or thermoelectrical heater). Due to the thin nature of the film, heat may be quickly transferred through the film into and out of the reaction chamber. This enables the temperature of a sample received within the reaction chamber to be varied at a high rate. Moreover, the thin nature of the film allows for the temperature of the sample to be accurately controlled and provides an improved distribution of temperature throughout the sample.

Pressurising the sample in the reaction chamber will also cause the sample to apply pressure against the flexible film. Indeed, the film is preferably configured to deform under the internal pressure within the reaction chamber. As such, the pressure within the reaction chamber may push the film outward, into contact with a thermal surface configured to heat and/or cool the contents of the reaction chamber. Consequently, the flexible film will take the shape of and conform to the thermal surface, enabling quick and uniform transfer of heat to a sample within the reaction chamber. Thus the temperature of the fluid sample can be more accurately controlled and varied, and the distribution of temperature across the fluid sample may be further improved.

The cartridge also reduces the risk of fluid contained in the reaction chamber from being accidentally released during or after use. Sealing the inlet and outlet port helps to prevent escape of a sample. Indeed, preferably the inlet and outlet ports are configured to be permanently (i.e. irreversibly) sealed so as to reduce the risk of sample escaping the cartridge during or after use.

During use, the air reservoir provides volume for fluid within the reaction chamber to thermally expand into as its temperature is increased. This is achieved since the gas stored or retained therein will typically have a higher compressibility than the fluid. This volume for expansion further reduces the risk that the cartridge will rupture or leak.

After thermal cycling the internal pressure within the cartridge may be safely reduced. As discussed above, the flexible film may deform outwards under internal pressure from within the reaction chamber. Therefore, after the cartridge is separated from the thermal surface, the internal pressure within the cartridge will tend to cause the flexible film to expand outwards or swell. The resulting increase in the volume of the reaction chamber causes a corresponding reduction of the pressure within the cartridge. This depressurisation further reduces the risk of leakage from the cartridge and allows for safe disposal.

Thus these sample analysis cartridges in accordance with the invention offer safer and more effective preparation of samples.

Sample analysis cartridges in accordance with the invention may be configured to be non-permanently inserted into or received in sample analysis system (e.g. a point-of-care diagnostic device) and are well suited for use in polymerase chain reaction (PCR) analysis. The cartridge may be further configured to receive the sample and/or reagents in further reservoirs or chambers other than the reaction chamber discussed above. Thus the cartridge may store the sample before it is introduced to the reaction chamber. In further embodiments the cartridge may comprise one or more reservoirs in which DNA/RNA extraction and purification may be performed to produce the sample before the sample is transferred into the reaction chamber.

Preferably the cartridge further comprises a frame defining the lateral extents of the reaction chamber, wherein the film is more flexible than the frame. This rigid frame protects the contents of the cartridge, reducing the chance of the leakage or escape of any sample within. The frame may comprise a plurality of walls which define the reaction chamber, ports, channels and air reservoir. In particularly preferred example the frame may be formed of a rigid plastic such as polypropelene (e.g. Purell RP374R manufactured by LyondellBasell®) or cyclic olefin copolymer (COC) (e.g. TOPAS COC 5013L-10 manufactured by TOPAS Advanced Polymers®). Cartridges of such materials are cheap and quick to manufacture.

In preferred examples at least a portion of the frame is colourless and/or transmissive to electromagnetic radiation. In particularly preferred examples one or more of the walls that define the extents of the reaction chamber may be transparent to electromagnetic radiation such as visible light, ultraviolet light and/or infrared light. Therefore, a sample within the reaction chamber may be irradiated with radiation transmitted through these walls, and radiation emitted by the sample may be collected after transmission out the reaction chamber through these walls. Internal and external surfaces of the frame and the walls discussed above may be polished so as to further improve their optical properties.

Preferably the inlet and outlet ports are permanently sealable. Thus preferably the inlet and outlet ports may be irreversibly closed so as to prevent any escape of a sample during use. In some examples the cartridges may comprise an inlet channel extending between the inlet port and the inlet of the reaction chamber and/or an outlet channel extending between the outlet port and the outlet of the reaction chamber. Thus the inlet and outlet channels may fluidly connect the reaction chamber to the respective port. The inlet channel and/or the outlet channel may be arranged such that they may be welded closed so as to seal the respective port. In preferred examples the inlet channel and/or outlet channel are arranged such that they may be welded closed at two positions separated along the length of the respective channel. This provides a highly effective seal since fluids such as the sample are unlikely to escape past two welds.

Indeed, the cartridge may comprise an inlet channel extending between the inlet port and an inlet of the reaction chamber, the inlet channel comprising two adjacent sealing portions that extend past one other, and/or an outlet channel extending between an outlet of the reaction chamber and the outlet port, the outlet channel comprising two adjacent sealing portions that extend past one another. For instance, the sealing portions of the inlet and/or outlet channel may be laterally separated by a distance that is less than ten times the width of the channel, more preferably less than five times the width of the channel. The two adjacent sealing portions of the respective channel may welded closed in a single step using a single heated element (e.g. a heat probe). Thus a highly effective seal comprising two welds that are separated along the length of the respective channel may be conveniently formed. In preferred examples, the inlet and/or outlet channel may double back on themselves, such that the sealing portions are separated along the length of the respective channel by a bend in the respective channel that is in the range 90 to 180 degrees, and preferably from 135 to 180 degrees such that the sealing portions are substantially parallel to one another. However, it will be appreciated that other arrangements are equally possible.

Alternatively or additionally, the inlet and/or outlet ports may be arranged to be closed by an adhesive and/or a cap or plug that blocks the respective port. Alternatively any other means of sealing the ports may be provided.

Preferably, a lateral dimension of the reaction chamber parallel to the plane in which the film extends is at least three times the thickness of the reaction chamber perpendicular to the plane in which the film extends, preferably at least five times the thickness, more preferably ten times the thickness. Thus preferably the reaction chamber is substantially planar, extending in a plane parallel to the film. This helps ensure that heat is rapidly transferred to and from the contents of the reaction chamber via the film and that the temperature of the contents the reaction chamber remains uniform throughout. For example, the reaction chamber may have an aspect ratio of between 10:1 and 20:1. For instance, the reaction chamber may have a thickness of from 1 to 2 mm, whereas the width and/or a length of the chamber may be in the range from 10 to 20 mm.

Preferably the cartridge further comprises: an excitation wall, the excitation wall forming a face of the reaction chamber and arranged to transmit excitation radiation into the reaction chamber; and, an emission wall, the emission wall forming a face of the reaction chamber and arranged to transmit emission radiation from the reaction chamber; wherein the excitation wall and emission wall are angularly offset, and wherein preferably the angle between the excitation wall and emission wall is at least 30 degrees, preferably at least 45 degrees, more preferably at least 60 degrees, more preferably still at least 75 degrees.

Such an angular offset between the emission wall and excitation wall through which radiation is respectively transmitted into and received from the reaction chamber provides significant improvements in accuracy and precision. The interference or interaction between the excitation and emission radiation is reduced such that the proportion of excitation radiation observed in the emission radiation is reduced. As such, the portion of the emission radiation which relates to the contents of the reaction chamber (e.g. radiation which has been reflected, scattered, transmitted, or emitted by the contents of the reaction chamber) may be more accurately and reliably identified and analysed.

In accordance with a further aspect of the invention there is provided a method for preparing a sample using a sample analysis cartridge, the cartridge comprising: a reaction chamber configured to receive a sample; a film defining at least a portion of a face of the reaction chamber; an inlet port fluidly connected to an inlet of the reaction chamber and an outlet port fluidly connected to an outlet of the reaction chamber; and an air reservoir fluidly connected to the reaction chamber; the inlet port, outlet port and air reservoir arranged such that as the reaction chamber is filled with fluid through the inlet port gas within the reaction chamber may escape the cartridge through the outlet port whilst gas within the air reservoir is retained; the inlet port, outlet port and air reservoir being further arranged such that, if the inlet and outlet ports are sealed, gas within the air reservoir applies pressure on the contents of the reaction chamber; wherein the method comprises: securing the cartridge to a thermal surface such that the film is adjacent to the thermal surface, the thermal surface being arranged to vary the temperature of the contents of the reaction chamber; a first filling step in which sample is dispensed via the inlet port so as to fill the reaction chamber, during which gas within the reaction chamber escapes the reaction chamber via the outlet port whilst gas within the air reservoir is retained within the air reservoir; sealing the outlet port; a second filling step in which further fluid is dispensed ivia the inlet port so as to pressurise the reaction chamber such that at least a portion of the film conforms to the thermal surface; sealing the inlet port; and, varying the temperature of the sample using the thermal surface.

Such a method offers improved preparation and analysis of samples. The method allows for the contents of the reaction chamber within the sample analysis cartridge to be quickly and effectively thermally cycled. The method enables the sample to be accurately analysed and safely disposed of. The method may be automated, and be performed without human interaction by a point of care diagnostic device. The method is suitable for use in a range of analytically techniques, including as part of a method for PCR analysis.

The method may be performed using any of the sample analysis cartridges discussed above with reference to the preceding aspect of the invention, and offer corresponding advantages.

To secure the cartridge the cartridge may be clamped to the thermal surface. Thus the method may comprise a preceding step in which the cartridge is first inserted into a sample analysis system.

However, in preferred examples securing the cartridge comprises securing the cartridge between two opposing thermal surfaces arranged to vary the temperature of the reaction chamber. Consequently the cartridge may be secured such that the flexible film is adjacent to a first thermal surface, whereas an opposing wall of the cartridge may be adjacent to the opposing second thermal surface. The temperature of the sample within the cartridge may subsequently be varied by simultaneously applying or removing heat from the film and the opposing wall using the thermal surfaces. Thus the temperature of sample within the reaction chamber may be varied more quickly and may have a more uniform temperature distribution during use.

The first filling step may be performed using a pipette or other dispensing equipment. Each filling step may comprise dispensing pre-determined amounts of fluid. For instance the first filling step may involve dispensing a volume of sample which is at least as great as the volume of the reaction chamber so as to entirely fill the reaction chamber. The second filling may comprise dispensing a smaller volume of fluid to pressurise the cartridge.

In preferred examples the sample dispensed into the reaction chamber during the first and/or second filling step may comprise a purified eluate containing DNA or RNA segments. For instance, the method may be performed as part of PCR analysis of such DNA and RNA segments. Consequently, in particularly preferred examples the method may additionally comprise extracting the DNA or RNA segments and/or purifying the DNA or RNA segments to produce the sample.

Following the second filling step the film may contact and conform to the adjacent thermal surface, such that at least a portion of the film is parallel to the thermal surface. This increases the rate of thermal transfer between the film and thermal surface. The second filling step preferably ensures that the pressure applied by the gas retained in the air reservoir is such that at least a portion of the film continues to conform to thermal surface whilst the temperature of the sample is varied. Thus the gas within the air reservoir acts as an energy store and ensures that good thermal contact is maintained between the film and the thermal surface during use. Thus the temperature of the sample may quickly and efficiently varied.

Preferably the second filling step involves dispensing further sample to pressurise the cartridge. Using additional sample rather than an alternative liquid offers the advantage that the sample dispensed in the first filling step is not diluted. Equally, using additional sample instead of a gas (e.g. air) means that the effects of pressure variations do not cause the sample to exit the reaction chamber as might occur if gases were retained both upstream of the reaction chamber (e.g. in an inlet channel) and downstream of the reaction chamber (e.g. in the air reservoir and/or the outlet channel). Nevertheless, in some embodiments a gas or liquid other than sample may be used to pressurise the reaction chamber.

Preferably sealing the inlet port and/or the outlet port comprises permanently sealing the respective port. Thus the port(s) may be irreversibly closed such that the contents of the cartridge cannot escape and the cartridge may be effectively pressurised. Preferably sealing the outlet port comprises welding closed an outlet channel extending between the outlet port and an outlet of the reaction chamber and/or sealing the inlet port comprises welding closed an inlet channel extending between the inlet port and an inlet of the reaction chamber. Alternatively, the inlet port and/or the outlet port may be closed using an adhesive and/or a cap or plug.

In particularly preferred examples the method comprises welding closed the outlet channel at two positions separated along the length of the channel and/or wherein sealing the inlet port comprises welding closed the inlet channel at two positions separated along the length of the channel. As discussed above, such seals are particularly effective at preventing fluids escaping from the reaction chamber. Thus the reaction chamber can be easily pressurised during the second filling step and sample within the reaction chamber is unlikely to escape or leak during or after use.

Preferably varying the temperature of the sample using the thermal surface comprises thermal cycling the sample. Thus the method is suitable for use in PCR techniques. In further examples the temperature of the sample is not cyclically varied (as occurs during thermal cycling), but rather the sample is simply heated or cooled.

Preferably the method comprises the further step of releasing the cartridge, such that the film expands outwards, reducing the pressure within the reaction chamber. Thereafter, the cartridge may subsequently be safely disposed of.

Optionally, the method may further comprise optical analysis of the sample. In preferred examples the method may comprise: irradiating the sample within the reaction chamber; and receiving radiation emitted from the reaction chamber. The emitted radiation detected in this manner may subsequently be analysed to detect features or content of the sample—e.g. the properties or identity of DNA or RNA segments within the sample.

Preferably the method further comprises irradiating the contents of the reaction chamber with excitation radiation from one or more sources, the excitation radiation being directed towards the sample analysis cartridge along a first path; and detecting emission radiation from the reaction chamber using the one or more detectors, the emission radiation being transmitted from the cartridge along a second path, wherein the first and second paths are angularly offset.

In accordance with a further aspect of the invention there is provided a sample analysis system configured to receive a cartridge as discussed above with reference to the preceding aspects of the invention, the system comprising: a thermal surface arranged to vary the temperature of the contents of the reaction chamber; a securing mechanism configured to detachably secure the cartridge to the thermal surface such that the film is adjacent to the thermal surface; a dispensing module configured to dispense sample into the inlet port of the cartridge; and a sealing mechanism configured to seal the outlet and inlet ports of the cartridge. Preferably the sample analysis system is a point-of-care diagnostic system, being configured to analyse a sample at the location of a patient to ensure rapid analysis and to improve overall care for the patient.

The system is configured to prepare a sample within a sample analysis cartridge as discussed above in reference to the first aspect of the invention—e.g. by thermal cycling the cartridge. Equally, the sample analysis system may be configured to perform any of the methods discussed above. Thus the system may comprise any of the optional or preferable features discussed above with reference to cartridges and methods in accordance with the invention and offer corresponding benefits and advantages. Notably, the system—which may be used for PCR—is accurate, convenient to use and is less prone to unintended leaks of samples. The system may be automated and may be small in size and weight, as well as being easy to manufacture and of low cost. The system may form part of a point of care medical diagnostic device.

The thermal surface may be a surface of heating blocks of a thermal cycler or a surface of a heater such as a resistive heater, a thin film heater, or a thermoelectrical cell (also commonly termed a Peltier cell). The sealing mechanism may comprise one or more heat probes. The heat probe(s) may be moveable and may be arranged to weld channels within the cartridge closed. Alternatively, the securing mechanism may comprise a movable cap or plug. The dispensing module preferably comprises a movable pipette which may be arranged to dispense pre-determined amounts of fluids such as a sample into the inlet port of the cartridge.

The sample analysis system may also be configured to be perform optical analysis of the sample. In preferred examples the sample analysis system further comprises an optical analysis module preferably comprising: a source module configured to emit excitation radiation into the reaction chamber of the cartridge; and, a detection module configured to receive emission radiation emitted from the reaction chamber. The radiation received by the optical analysis module may subsequently be analysed to determine the contents of the reaction chamber and/or parameters of these contents. Following this analysis the cartridge may be removed from the system for safe disposal.

Preferably the system comprises one or more sources configured to irradiate the reaction chamber of a sample analysis cartridge received by the diagnostic system within excitation radiation, the excitation radiation being directed towards the sample analysis cartridge along a first path; and one or more detectors configured to receive emission radiation from the reaction chamber, the emission radiation being emitted from the sample analysis cartridge along a second path; wherein the first and second paths are angularly offset. The sample analysis system may be further configured to perform any of the optional or preferable steps discussed above with reference to the preceding aspects of the invention.

According to a further aspect of the invention there is provided a system comprising a sample analysis system and a cartridge as discussed with reference to the preceding aspects of the invention. The sample analysis system and cartridge may comprise any of the optional and/or preferable features discussed above and offers corresponding advantages.

In accordance with a further aspect of the invention there is provided a point-of-care diagnostic system configured to receive a sample analysis cartridge, the system comprising: one or more sources configured to irradiate the reaction chamber of a sample analysis cartridge received by the diagnostic system within excitation radiation, the excitation radiation being directed towards the sample analysis cartridge along a first path; and one or more detectors configured to receive emission radiation from the reaction chamber, the emission radiation being emitted from the sample analysis cartridge along a second path; wherein the first and second paths are angularly offset.

Such a point-of-care diagnostic system may comprise any of the features of the sample analysis systems discussed above with reference to the preceding aspect of the invention. Similarly, in preferred examples the sample analysis systems may be configured to perform any of the method steps discussed above.

In preferred examples, the point-of-care diagnostic systems discussed above are configured to receive any of the sample analysis cartridges discussed above with reference to the earlier aspects of the invention. However, this is not essential, and the point-of-care diagnostic systems may be configured to receive alternative cartridges, such as cartridges without the air reservoirs, ports and/or films discussed above.

Point-of-care diagnostic systems in accordance with the invention offer quick, efficient and accurate optical analysis of samples within a sample analysis cartridge. In particular, properties of the contents of the reaction chamber may be quickly and efficiently determined using signals received by the one or more detectors when the contents are exposed or irradiated with radiation from the one or more sources. The manner in which a sample reflects, transmits, absorbs, refracts or scatters radiation (e.g. light) may provide information regarding the properties and/or identity of the sample. Consequently, these systems are well suited for analysing or interrogating samples after they have been thermal cycled—e.g. DNA samples that have been amplified using PCR techniques.

An improvement in accuracy and precision is achieved by the specific arrangement of sources and detectors discussed above because interference or interaction between the excitation and emission radiation is reduced. In other words, the proportion of excitation radiation observed in the emission radiation is reduced. As such, the portion of the emission radiation which relates to the contents of the reaction chamber (e.g. radiation which has been reflected, scattered, transmitted, or emitted by the contents) may be more accurately and reliably identified and analysed.

In contrast, if the path of the excitation radiation emitted into the reaction chamber were to be aligned with the path of the emission radiation exiting the reaction chamber (e.g. if the first and second paths were to be collinear), the emission radiation received by the detectors may contain significant amounts of unchanged excitation radiation that has been transmitted straight through the reaction chamber from the sources to the detectors. As such, when analysing the detection results from the detectors, it can be difficult or complex to isolate the portion of the excitation radiation which relates to the contents of the reaction chamber (e.g. radiation emitted, scattered, refracted or absorbed by the contents of the reaction chamber) from the relatively large amounts of emission radiation contained in the excitation radiation.

Thus, the angular offset discussed above improves accuracy and precision of analysis of emission radiation received from the reaction chamber since it increases the proportion of information in the emission radiation which relates to the contents of the reaction chamber. Furthermore, the computational resources and time required to isolate the information relating to the contents of the reaction chamber in the emission radiation from the information relating to the excitation emission is reduced.

Herein the term “radiation” will be understood as electromagnetic radiation, rather than other forms of radiation such as heat or particle. As such, the radiation emitted by the one or more sources and received by the one or more detectors may comprise light of visible, infrared and/or ultraviolet wavelengths, as well as radiation of other wavelengths outside these ranges.

By “angularly offset” it is understood that the first and second paths are not parallel or substantially parallel, being skewed relative to one another. Preferably the angle between the first and second paths is at least 30 degrees, preferably at least 45 degrees, more preferably at least 60 degrees, more preferably still at least 75 degrees. Increasing the angular offset can reduce the interaction between the excitation and emission radiation—i.e. reducing the proportion of the emission radiation which is formed of excitation radiation—improving the accuracy of analysis performed using the system. In particularly preferred examples the first and second paths are substantially perpendicular. As such, the first and second paths may be arranged at an angle of approximately 90 degrees.

Preferably the system is configured to non-permanently receive sample analysis cartridges, such that different sample analysis cartridges may be inserted and removed from the system as required (e.g. to analyse different samples).

Preferably the first path extends through an excitation wall of the reaction chamber and the second path extend through an emission wall of the reaction chamber, the excitation wall and emission wall being different. This arrangement may further reduce the amount of excitation radiation present in the emission radiation since the one or more detectors are less likely to receive excitation radiation that has reflected from the walls of the reaction chamber without interacting with the contents of the reaction chamber (e.g. a sample within the reaction chamber). Preferably the system is configured such that the first path is perpendicular or substantially perpendicular to the excitation wall, and the second path is perpendicular or substantially perpendicular to the emission wall. Preferably the excitation wall and emission wall are angularly offset from one another.

In particularly preferred examples the system further comprises an optical analyser configured to analyse the emission radiation received by the detectors. As such, the optical analyser may be configured to determine properties of the contents of the reaction chamber using detection results or signals received from the one or more detectors. For instance, the optical analyser may determine the identity of a sample within the reaction chamber (e.g. the type of DNA within a sample that has been amplified by PCR). However, alternatively, an optical analysis module may be provided separately from the system. In either case, the one or more detectors may be configured to send a detection result (e.g. a signal) corresponding to the excitation radiation received by said detectors to the optical analysis module. The optical analyser may be configured subsequently analyse the detection result received from the one or more detectors to determine properties of the contents of the reaction chamber.

In particularly preferred examples the system comprises a plurality of sources each configured to emit radiation of different properties, wherein preferably said different properties comprises one or more of different wavelengths, polarisations, phases, coherences and/or amplitudes. As such, the system is preferably configured to irradiate the contents of the reaction chamber with a variety of different radiations. Differences and similarities of the emission radiation emitted from the reaction chamber under each of these radiations may offer further information regarding the contents of the reaction chamber. As such, the variety of analytical techniques may be increased through the use of a plurality of sources which each emit radiation of respective properties. For instance, the system may comprise sources configured to emit visible light at different wavelengths (i.e. with different colours), or light in different regions of the electromagnetic spectrum (e.g. the visible, UV or IR regions).

More preferably the system may comprise a beam combining structure configured to direct radiation emitted by the plurality of sources along the first path towards the sample analysis cartridge. As such, radiation from each source or multiple sources in combination can be directed to the sample analysis cartridge along the first path. This offers a particularly compact structure. Alternatively, the system may be configured to direct the radiation emitted by the plurality of sources to the sample analysis cartridge along a corresponding plurality of first paths, each of which is angularly offset from the second path. For instance, each of the plurality of first paths may be parallel and/or arranged in a single plane, whereas the second path to the detectors may be arranged out of this plane such that the second path is angularly offset from each of the first paths. In further examples the system may comprise a single source configured to selectively emit radiation having different properties. Each of these arrangements increases the flexibility of the system since the radiation incident on the contents of the reaction chamber can be varied, increasing analysis possible from the detection results or signals output by the detectors.

In particularly preferred examples the system comprises a plurality of detectors each configured to detect radiation of different properties, wherein preferably said different properties comprise one or more of different wavelengths, polarisations, phases, coherences and/or amplitude. As such, each detector may be configured to detect radiation of a respective property or properties. For instance, the system may comprise visible light, UV and IR detectors. As such, analysis of the contents of the reaction chamber in the may be based on detection results comprising information regarding different properties of the emission radiation. As such, flexibility of the system may be improved and the range of analytical techniques increased.

Preferably the system comprises a beam splitting structure configured to split the emission radiation into a corresponding plurality of beams and to direct each beam to a corresponding detector. This is a particularly compact approach, but is not essential. Alternatively, the system may comprise a single detector configured to detect radiation having a range of properties.

Preferably the system comprises a collimator configured to receive emission radiation from the sample analysis cartridge and to form the received emission radiation into a beam. For instance the system may comprise a collimating lens. The focused beam of emission radiation is easier to detect and analyse. The system may be further configured to direct this beam of emission radiation to the one or more sources, potentially via the beam splitting structure discussed above.

Preferably the system comprises a dispensing mechanism configured to introduce a sample into the reaction chamber and/or a sealing mechanism configured to seal the reaction chamber so as to prevent the escape of the contents of the reaction chamber. As discussed above, the dispensing mechanism may comprise a pipette configured to dispense a sample into the reaction chamber. The dispensing mechanism may be configured to dispense a sample into the reaction chamber from another reservoir or chamber within the sample analysis cartridge. The sealing mechanism may be a heat probe configured to weld closed a port of the sample analysis cartridge through which a sample may be inserted. As previously discussed, sealing the sample analysis cartridge may reduce the chance that a sample or other contents escapes the cartridge, contaminating the system and/or its surroundings. Indeed, preferably the inlet and outlet ports are configured to be permanently (i.e. irreversibly) sealed so as to reduce the risk of sample escaping the cartridge during or after use. Using such equipment the system may automate filling and/or sealing of the reaction chamber.

Preferably the system further comprises a thermal surface that is configured to heat and/or cool the contents of the reaction chamber. Preferably the thermal surface is configured to thermal cycle the contents of the reaction chamber. As such, the system may be well suited to perform PCR analysis. For instance the thermal surface may be a surface of heating blocks within a thermocycler, and/or the surface of a heater (e.g. a resistive, thin film or thermoelectrical heater).

In preferred examples the system further comprises a securing mechanism configured to detachably retain the sample analysis cartridge against the thermal surface. Securing the sample analysis cartridge in this manner may ensure good thermal contact between the thermal surface and the contents of the reaction chamber. This allows for the temperature of the contents of the reaction chamber (e.g. a sample) to be accurately controlled and varied at a high rate.

Preferably the system comprises a controller configured to operate the one or more sources and one or more detectors. In further examples the controller may be further configured to operate any of the optical analysers, dispensing mechanisms, sealing mechanisms and thermal surfaces discussed above. The controller may comprise one or more processors, microprocessors, computers or other controllers configured with programmed instructions to operate the systems discussed above.

According to a further aspect of the invention there is provided a sample analysis cartridge for use in a point-of-care diagnostic system according to the previous aspect of the invention, the cartridge comprising a reaction chamber configured to receive a sample; an excitation wall, the excitation wall forming a face of the reaction chamber and arranged to transmit excitation radiation into the reaction chamber; and, an emission wall, the emission wall forming a face of the reaction chamber and arranged to transmit emission radiation from the reaction chamber; wherein the excitation wall and emission wall are angularly offset. Such sample analysis cartridges may be inserted into point-of-care diagnostic systems for rapid and accurate analysis of their contents.

Preferably sample analysis cartridges in accordance with this aspect invention are configured to be non-permanently inserted into or received in any of systems discussed above (e.g. a point-of-care diagnostic system of the previous aspect) and are well suited for use in polymerase chain reaction (PCR) analysis. The cartridge may be further configured to receive the sample and/or reagents in further reservoirs or chambers other than the reaction chamber discussed above. Thus the cartridge may store the sample before it is introduced to the reaction chamber. In further embodiments the cartridge may comprise one or more reservoirs in which DNA/RNA extraction and purification may be performed to produce the sample before the sample is transferred into the reaction chamber.

The provision of a cartridge having a reaction chamber with angularly offset walls that are transmissive to electromagnetic radiation (e.g. optically transmissive) allows for improvements in the analysis of the contents of the reaction chamber. Specifically, by offsetting transmissive walls through which radiation may be supplied to and received from the reaction chamber the interference between the excitation and emission radiation can be reduced. This can improve the accuracy of, and reduce the resources required for analysis of, emission radiation detected from the reaction chamber. In preferred examples the emission wall and excitation wall are substantially perpendicular, although this is not essential.

The excitation wall and emission wall define at least a portion of the boundary of the reaction chamber. The excitation and emission walls may also form part of a larger frame of the sample analysis cartridge. This frame may additionally comprise a plurality of walls which define the remaining boundaries of the reaction chamber, as well as ports and channels through which a sample may be introduced into the reaction chamber, and further chambers or reservoirs (as discussed above). The frame may be rigid, such that it is capable of withstanding typical stresses applied to the cartridge during standard use.

Preferably the emission wall and/or excitation wall is colourless and/or substantially transparent over at least a portion of the electromagnetic spectrum. In particularly preferred examples the emission wall and/or excitation wall may be transparent to visible light, ultraviolet light and/or infrared light. Therefore, a sample within the reaction chamber may be easily irradiated with radiation transmitted through the excitation wall (e.g. by the one or more sources of the systems discussed above). Whereas, radiation from the sample—such as radiation reflected, emitted, scattered or transmitted by the sample—may be easily detected (e.g. by the one or more detectors of the systems discussed above) after transmission out the reaction chamber through the emission wall. Preferably the internal and external surfaces of the emission and excitation walls may be polished so as to further improve their optical properties. The remaining portions of the frame may exhibit the same properties.

In particularly preferred example the emission and excitation walls (and the remainder of a wider frame) may be formed of a rigid plastic such as polypropelene (e.g. Purell RP374R manufactured by LyondellBasell®) or cyclic olefin copolymer (COC) (e.g. TOPAS COC 5013L-10 manufactured by TOPAS Advanced Polymers®). Cartridges of such materials are cheap and quick to manufacture and have good optical transmission properties. Moreover, a rigid frame is resistant to damage.

Preferably the area of the emission wall is greater than the area of the excitation wall, preferably at least three times greater, more preferably at least five times greater, more preferably still at least ten times greater. As such, the aspect ratio between these walls of the reaction chamber is high. Detection of radiation from the contents of the reaction chamber is simplified by increasing the relative size of the emission wall since more radiation may be transmitted from the reaction chamber and directed to a detector (e.g. the one or more detectors of the systems discussed above).

According to a further aspect of the invention there is provided a system comprising a point-of-care system as discussed above with reference to the first aspect of the invention and one or more sample analysis cartridges in accordance with the previous aspect of the invention. This system may comprise any of the optional or preferable features discussed above with reference to these systems and cartridges and offer corresponding benefits.

According to a further aspect of the invention there is provided a method using the system according to the first aspect of the invention discussed above, the method comprising: inserting a sample analysis cartridge into the point-of-care diagnostic system, the sample analysis cartridge comprising a reaction chamber; irradiating the contents of the reaction chamber with excitation radiation from the one or more sources, the excitation radiation being directed towards the sample analysis cartridge along a first path; detecting emission radiation from the reaction chamber using the one or more detectors, the emission radiation being transmitted from the cartridge along a second path.

This method offers corresponding advantages and benefits to the systems and devices discussed above. In particular the method provides improvements in accuracy and efficiency in optical analysis of samples, especially DNA samples which have previously been extracted, purified and amplified using PCR techniques. In such cases the sample may be a purified eluate comprising DNA. The method is well suited for use as a point-of-care analysis.

The method may be performed using any of the sample analysis cartridges discussed above with reference to the preceding aspects of the invention.

Preferably the method comprises analysing the emission radiation to detect properties of the contents of reaction chamber. This analysis may be performed with an optical analyser or any other suitable equipment as discussed above.

Preferably the method comprises: introducing a sample into the reaction chamber of the sample analysis cartridge, and/or sealing the reaction chamber of the sample analysis cartridge to prevent the contents of the reaction chamber escaping. The introduction of sample into the reaction chamber may be performed with a pipette or other dispensing mechanism, as discussed above. The sample may be transferred into the reaction chamber from a further chamber or reservoir within the sample analysis cartridge (e.g. a chamber or reservoir in which the sample has previously been extracted or purified by the system). Sealing may be performed using the sealing mechanism discussed above. For instance, sealing the reaction chamber may involve sealing an inlet port and/or the outlet port through which a sample may introduced into the reaction chamber. Preferably sealing the outlet port comprises welding closed an outlet channel extending between the outlet port and an outlet of the reaction chamber and/or sealing the inlet port comprises welding closed an inlet channel extending between the inlet port and an inlet of the reaction chamber. Alternatively, the inlet port and/or the outlet port may be closed using an adhesive and/or a cap or plug. Advantageously these approaches permanently (i.e. irreversibly) seal or close the reaction chamber, preventing escape of sample after analysis.

Preferably the cartridge is secured against a thermal surface using a securing mechanism. This offers good thermal transfer between the thermal surface and the cartridge. For instance, the cartridge may be detachably clamped to the thermal surface using a clamp. In other examples securing the cartridge comprises securing the cartridge between two opposing thermal surfaces arranged to vary the temperature of the reaction chamber. The temperature of the sample within the cartridge may subsequently be varied by simultaneously applying or removing heat using opposed the thermal surfaces. Thus the temperature of sample within the reaction chamber may be varied more quickly and may have a more uniform temperature distribution during use.

Preferably, the sample analysis cartridge is inserted into the point-of-care diagnostic system such that the cartridge is in thermal contact with a thermal surface, and the method further comprises: thermal cycling the contents of the reaction chamber. Thus the method is suitable for use in PCR techniques. In further examples the temperature of the sample is not cyclically varied (as occurs during thermal cycling), but rather the sample is simply heated or cooled.

After analysis the method may further comprise removing the sample analysis cartridge from the point-of-care diagnostic system, and disposing of the cartridge.

### BRIEF DESCRIPTION OF DRAWINGS

Specific examples of the invention will now be discussed with reference to the following drawings:

FIG. 1a shows a plan view of a sample analysis cartridge according to an embodiment of the invention;

FIG. 1b shows a cross section through the sample analysis cartridge shown in FIG. 1a;

FIG. 2 provides a flow chart of a method according to an embodiment of the invention, each step of the method is illustrated with schematic views of a sample analysis cartridge and sample analysis system according to embodiments of the invention, the figure further comprises a graph schematically illustrating the variation of pressure within the sample analysis cartridge during the method;

FIG. 3a shows a plan view of a portion of a further sample analysis cartridge according to the invention;

FIGS. 3b and 3b show cross sections through the sample analysis cartridge shown in FIG. 1b;

FIG. 3d shows a further plan view of a portion the sample analysis cartridge shown in FIG. 3a and illustrates where heat may be applied to the sample analysis cartridge to seal a reaction chamber of the cartridge; and

FIG. 4 schematically shows a system for optical analysis of the content of a sample analysis cartridge according to an embodiment of the invention.

### DETAILED DESCRIPTION

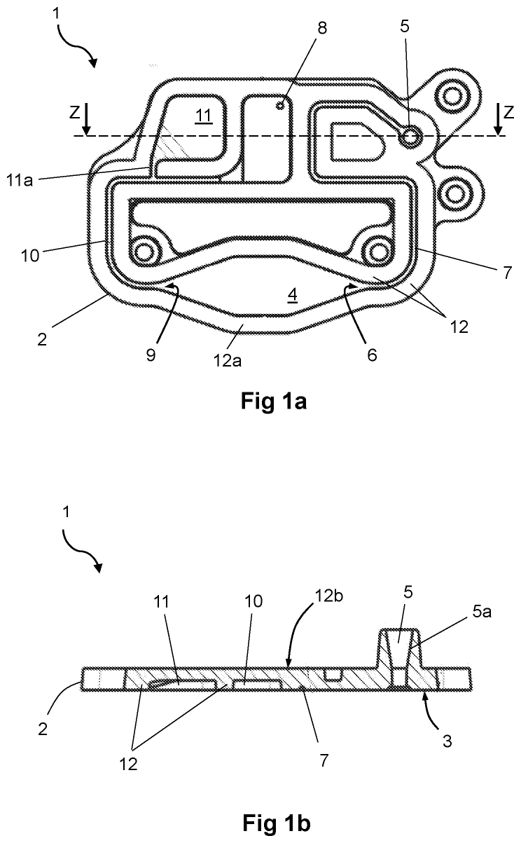

An example sample analysis cartridge 1 is shown in plan view in FIG. 1a. A cross section through the cartridge 1 along line Z-Z is shown in FIG. 1b.

The sample analysis cartridge 1 comprises a rigid frame 2 and a flexible film 3. The term “film” will be understood to be a thin, flexible sheet of material. Such flexible films may include a variety of materials including membranes and foils.

The frame 2 defines a plurality of walls 12. A reaction chamber 4 is defined within the walls 12 of the frame 2 and the film 3. The reaction chamber 4 is arranged to receive and contain a fluid (e.g. a sample containing DNA or RNA for amplification). The film 3 defines a face of the reaction chamber 4. Specifically, the film 3 defines the lower face of the reaction chamber 4 as shown in the cross section of FIG. 1b. Heat may quickly and efficiently transferred into and out of the reaction chamber 4 (e.g. during thermal cycling) via the film 3.

Due to the thin nature of the film 3, heat may be quickly transferred through the film into and out of the reaction chamber 4. This enables the temperature of a sample received within the reaction chamber 4 to be varied at a high rate. Moreover, the thin nature of the film 3 allows for the temperature of a sample within the reaction chamber 4 to be accurately controlled and provides an improved distribution of temperature throughout the sample.

The cartridge 1 further comprises an inlet port 5 through which fluids (e.g. a sample) can be introduced into the reaction chamber 4. The inlet port 5 is fluidly connected to an inlet 6 of the reaction chamber 4 by an inlet channel 7. Thus fluids may pass from the inlet port 5 to the inlet 6 of the reaction chamber 4 via the inlet channel 7. The cartridge 1 also further comprises an outlet port 8 through which fluids such as gases may leave or exit the reaction chamber 4. The outlet port 8 is fluidly connected to an outlet 9 of the reaction chamber 4 by an outlet channel 10. As such, fluids may pass from the outlet 9 of the reaction chamber 4 to the outlet port 8 via the outlet channel 10.

An air reservoir 11 is defined within the frame 12 of the cartridge 1. This air reservoir 11 is a void within the frame 12 comprising a single opening 11a that allows the air reservoir to communicate with the outlet channel 10 at a position between the outlet port 8 and the outlet 9 of the reaction chamber 4. Thus the air reservoir 11 is fluidly connected to the reaction chamber 4 and the outlet 9 of the reaction chamber 4 by the outlet channel 10. Having said this it will be appreciated that the arrangement of the air reservoir 11 shown in FIG. 1 is not essential and in further examples an air reservoir could be arranged to open to the reaction chamber 4 and/or the inlet channel 7 via an opening that is sufficiently narrow to prevent a fluid displacing gas retained therein whilst the reaction chamber 4 is being filled.

The air reservoir 11 is configured to retain gases (e.g. air) as the reaction chamber 4 is filled using the inlet port 5. The gas trapped within the air reservoir 11 in this manner may apply pressure on the contents of the reaction chamber 4.

Indeed, if the inlet and outlet ports 5, 8 are sealed after the reaction chamber 4 is filled with fluid, the stored or trapped gas (e.g. air) within the air reservoir 11 may exert pressure on the fluid (e.g. a sample) within the reaction chamber 4. Indeed, it will be appreciated that the gas within the air reservoir 4 acts as an energy store or “air-spring”, continuing to apply pressure to the fluid within the reaction chamber 11 as the temperature of the cartridge and the reaction chamber is varied—e.g. during thermal cycling. Pressurising the fluid in this manner acts to prevent outgassing, in which gas might otherwise come out of solution during heating. Thus the accuracy and sensitivity of optical analysis performed on a sample within the reaction chamber 4 after heating and/or thermal cycling may be increased.

The flexible film 3 is configured to deform under internal pressure within the reaction chamber 4. Therefore, the pressure applied by the air reservoir 11 may force the film 3 into conformance with an adjacent thermal surface (e.g. a surface used to heat the contents of the reaction chamber as discussed further below). Consequently, the flexible film 4 may take the shape of and conform to an adjacent thermal surface, enabling quick and uniform transfer of heat to a sample within the reaction chamber 4. Thus the temperature of a fluid sample can be more accurately controlled and varied, and the distribution of temperature across the fluid sample may be further improved.

Equally, without the support of an adjacent surface the flexible film 3 is configured expand or bow outwards when the internal pressure within the reaction chamber 4 is greater than the ambient pressure. This deformation in the shape of the film 4 will result in an increase in the internal volume of the reaction chamber 4 and reduce the pressure within the cartridge 1. This depressurisation which can occur after the cartridge is removed from a sample analysis system (e.g. a point of care diagnostic system) reduces the risk of leakage from the cartridge 1 and allows for safe disposal.

The walls 12 and the film 4 of the cartridge 1 are preferably optically transmissive such that they may transmit electromagnetic radiation into and out of the reaction chamber 4. Thus a sample received within the reaction chamber 4 may be optically analysed without being removed from the cartridge 1.

The cartridge 1 comprises an excitation wall 12a and an emission wall 12b that form angularly offset faces of the reaction chamber 4. Specifically, the excitation and emission walls 12a, 12b are perpendicular to one another. The emission wall 12b forms an upper face of the reaction chamber 4 as shown in FIG. 1b extending parallel to the film 3, whereas the excitation wall 12a defines a side face of the reaction chamber 4 (as seem most clearly in FIG. 1a).

The excitation and emission walls 12a, 12b are optically transmissive so that they may transmit electromagnetic radiation into and out of the reaction chamber 4. For instance, in preferred examples the excitation wall 12a and/or the emission all 12b may be substantially transparent to visible light, ultraviolet light and/or infrared light. The internal and external surfaces of the excitation and emission walls 12a, 12b (and the remaining walls 12 of the frame 2) may be polished so as to increase their optical transmissivity.

The inlet port 5 is raised from an upper surface of the cartridge 1 (and the emission wall 12b), as shown in FIG. 1b. The inlet port 5 is tapered, having a conical internal surface 5a, such that the inlet port 5 is wider at its distal end away from the cartridge 1 than at its proximal end at which the inlet port 5 communicates with the inlet channel 7. The tapered form of the inlet port 5 allows the reaction chamber to be easily filled—e.g. by a manual or automated pipette. Specifically, the conical internal surface 5a is arranged at an angle of 20 degrees relative to a direction in which the inlet port 5 extends away from the cartridge 1 (although in further examples the conical internal surface 5a may be arranged at angles between 10 and 45 degrees relative to this direction).

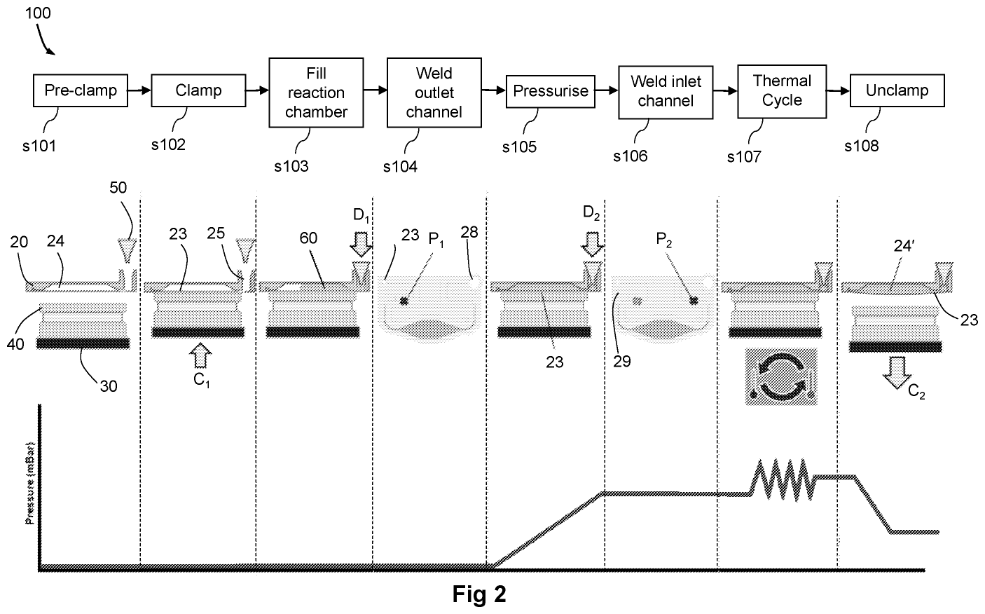

FIG. 2 illustrates a method 100 of preparing a sample within a sample analysis cartridge 20 using a sample analysis system. The cartridge 20 comprises corresponding features and offers similar benefits to the cartridge 1 discussed above with reference to FIGS. 1a and 1b. This method may form part of PCR analysis (although this is not essential).

As shown, the sample analysis system comprises a thermal surface 40 configured to vary the temperature of the contents of a cartridge 20 received with the system, a securing mechanism 30 configured to secure the cartridge 20 in contact with the thermal surface 40, and a dispensing module comprising a movable pipette 50 configured to engage an inlet port 25 of the cartridge 20 and to dispense fluids through the inlet port 25 so as to fill the reaction 24. The thermal surface 40 may be formed as part of a larger heating system.

In an initial pre-clamp step s101 the sample analysis cartridge 20 is inserted into the sample analysis system. As shown, the pressure within the reaction chamber 20 is low since the reaction chamber 20 fluidly communicates with the surrounding atmosphere via the inlet and outlet ports 25, 28 of the cartridge 20. Subsequently, in step s102 the securing mechanism 30 is operated to raise the thermal surface 40, as shown by arrow C₁. Thus the cartridge 20 is clamped against the thermal surface 40, securing the cartridge 20 such that a flexible film 23 that defines a face of a reaction chamber 24 of the cartridge 20 is adjacent to the thermal surface 40.

In step s103 the pipette 50 is inserted into the inlet port 25 and sample 60 is dispensed into the cartridge 20 as shown by arrow D₁. During this first filling step, as the liquid sample 60 enters the cartridge 20, air previously within the reaction chamber 24 is expelled from the cartridge 20 via the outlet port 28. However, air is retained within an air reservoir 29 which is formed as a void within the cartridge 20 that is fluidly connected to the reaction chamber 24 by a single opening, but lacks any further outlet. This occurs because the pressure within the cartridge 20 remains low since the reaction chamber 24 remains in fluid communication with the outside atmosphere via the outlet port 28. The pipette 50 may be arranged to dispense a known or predetermined volume of sample 60 into the cartridge in this step. For instance, the system may be arranged to dispense a volume of sample 60 that is equal to or greater than the volume of reaction chamber 24 using the pipette 50.

Subsequently, the outlet channel connecting the outlet port 28 to the reaction chamber 24 is welded closed at position P₁ in step s104. This permanently seals the outlet port 28, preventing fluids or gases from exiting the cartridge via the outlet port. The welding is preferably performed by a heat probe (not shown) that forms part of a sealing mechanism (again not shown) of the sample analysis system. The reaction chamber 24 of the cartridge 20 is then pressurised in step s105 as further sample 60 is dispensed into the reaction chamber 24 through the inlet port 25 by the pipette 50 (as shown by arrow D₂). This second filling step causes the internal pressure within the cartridge 20 to rise as the air retained within the air reservoir 29 is compressed. This increase in pressure is illustrated in the graph of FIG. 2. Consequently the pressure within the reaction chamber 24 is increased over ambient conditions. The increase in internal pressure causes the flexible film 23 to deform and to move into contact with the adjacent thermal surface 40. As such, the flexible film 23 conforms to and takes the shape of the thermal surface 40, ensuring rapid thermal transfer between the film 23 and the thermal surface 40. As a result there is also good thermal transfer between the sample 20 within the reaction chamber 24 and thermal surface 40.

Following the pressurisation step s105 an inlet channel that fluidly connects the inlet port 25 to the reaction chamber 24 is welded closed at position P₂ in step s106. The inlet port 25 is therefore permanently sealed, preventing the sample or air within the cartridge 20 from escaping. The air stored at pressure within the air reservoir 29 provides an energy store, acting as an “air-spring”, and ensures that the flexible film 24 continues to conform to the thermal surface 40. The welding process is again performed using a heat probe (not shown) which preferably forms part of sealing mechanism (not shown) of the sample analysis system. In some examples the same heat probe may be used to weld both the outlet channel and the inlet channel, in which case the heat probe may be moveable. However, this is not essential and the different channels may also be welded by separate heat probes.

The thermal surface 40 is subsequently operated to thermal cycle the sample 60 in step s107. The temperature of the sample 60 is cyclically raised and lowered using the thermal surface 40. This process may amplify DNA or RNA contained within the sample 60. The cyclical variation of temperature results in a cyclical variation in the pressure within the cartridge 20 as shown in the graph of FIG. 2. Throughout the thermal cycling process the high internal pressure ensures that the flexible film 24 continues to conform to the thermal surface 40. In addition the high internal pressure prevents the creation of bubbles—i.e. outgassing—within the sample 60.

After thermal cycling is complete, the cartridge 20 may be released by the securing mechanism 30 in steps 108. The cartridge 20 is unclamped from the thermal surface 40 and can be removed from the sample analysis system. As the cartridge 20 is released and separated from the thermal surface 40 the flexible film 23 may deform under the internal pressure within the cartridge 20 (which is high relative to the ambient pressure outside the cartridge). Thus the flexible film 23 expands outwards under pressure from the sample 60, indeed as will be seen in the schematic step s108 shown in FIG. 2 the flexible film is blown out, being deformed into a convex shape. The resulting expanded reaction chamber 24′ has a greater volume than the original form of the reaction chamber 24 before the start of the sample preparation process. This expansion in the size of the reaction chamber 24′ causes the pressure within the cartridge 20 to drop to a lower residual level, as shown in the graph of FIG. 2. This residual level is suitable for disposal of the cartridge 20 and is unlikely to result in any release or leakage of the sample 60 within the cartridge 20. Thus the method may comprise a final step of disposing of the cartridge 20. This disposal may be performed after the sample within the reaction chamber 24 has been analysed.

In further examples the sample analysis system may comprise two opposed thermal surfaces, and the cartridge 20 may be secured (e.g. clamped) between the two opposed thermal surfaces. As such, the flexible film 24 may be adjacent to a first thermal surface whereas a wall of the cartridge 20 which defines the opposing face of the reaction chamber 24 may be adjacent to the opposing second thermal surface. As such, the temperature of a sample 60 within the reaction chamber 24 may be controlled more quickly and may maintain a more uniform temperature throughout the preparation process.

In further examples the method may further incorporate optically analysing a sample 60 within a cartridge 20—e.g. after the sample has been thermal cycled in step s107. This optical analysis may be performed by an optical analysis module within the sample analysis system. The optical analysis module may be configured to emit excitation radiation to the reaction chamber 24 and to receive radiation emitted from the reaction chamber 24 (e.g. radiation which has been reflected, refracted, transmitted or emitted by a sample within the reaction chamber 24).

The optical analysis step may be performed whilst a cartridge 20 is secured or clamped to the thermal surface 40 by the securing mechanism or after the cartridge has been released by the securing mechanism 30 in step s108. In the latter case the analysis may be performed in-situ whilst the cartridge 20 is retained within the sample analysis system (although this is not essential).

The sample dispensed into the reaction chamber 24 during step s103 is preferably dispensed by the pipette 50 from a further storage chamber (not shown) within the cartridge 20. Where this method is used as part of PCR analysis, the sample introduced into the reaction chamber 24 in step s103 may comprise DNA or RNA segments. For instance, the sample may be a purified eluate containing DNA or RNA segments. Thus the method may further comprise preliminary steps of extracting and purifying DNA or RNA segments. Such steps are preferably performed by the sample analysis system and may be conducted within one or more further chambers (again not shown) of the cartridge 20. However, this is not essential.

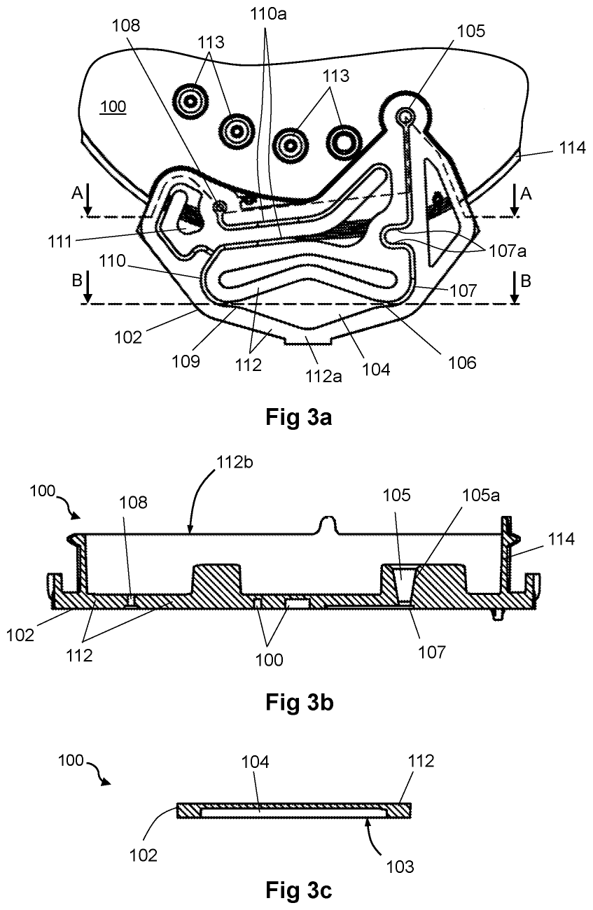

FIGS. 3a to 3d show a further exemplary sample analysis cartridge 100. A portion of the cartridge 100 is shown in plan view in FIGS. 3a and 3d, whereas cross sections of the cartridge 100 along lines A-A and B-B are shown in FIGS. 3b and 3c respectively.

This cartridge 100 shares many features and advantages with the sample analysis cartridge 1 shown in FIGS. 1a and 1b. Corresponding features shared between the two cartridges 1, 100 have had their reference signs incremented by 100 between the figures.

The cartridge 100 is generally circularly shaped, comprising a circular rim 114 of which a portion is shown n FIGS. 3a and 3b. As in the example shown in FIGS. 1a and 1b, the sample analysis cartridge 100 of FIG. 3 comprises a rigid frame 102 having a plurality of walls 112 and a flexible film 103. The rigid frame 102 is provided at the bottom position of the circular rim 114 shown in FIGS. 3a and 3d. A reaction chamber 4 is defined between the walls 112 and the film 103, and is configured to receive and contain a sample or other fluid. The film 104 defines a bottom face of the reaction chamber 4 (as seen most clearly in FIG. 3c).

The cartridge 100 further comprises an inlet port 105 through which fluids (e.g. a sample) can be introduced into the reaction chamber 104. The inlet port 105 is fluidly connected to an inlet 106 of the reaction chamber 104 by an inlet channel 107. The cartridge 100 also further comprises an outlet port 108 through which fluids such as gases may leave or exit the reaction chamber 104. The outlet port 108 is fluidly connected to an outlet 109 of the reaction chamber 104 by an outlet channel 110.

An air reservoir 11 is also defined within the frame 112 of the cartridge 100. This air reservoir 111 is a void within the frame 112 which fluidly communicates with the outlet channel 110 at a position between the outlet port 108 and the outlet 109 of the reaction chamber 104. Thus the air reservoir 111 is fluidly connected to the reaction chamber 104 and the outlet 109 of the reaction chamber 104 by the outlet channel 110. The air reservoir 111 is configured to retain gases (e.g. air) as the reaction chamber 104 is filled using the inlet port 105. The gas trapped within the air reservoir 111 in this manner may apply pressure on the contents of the reaction chamber 104.

The flexible film 103 is configured to deform under internal pressure within the reaction chamber 104. Thus if the pressure within the reaction chamber 104 is increased over the ambient pressure (e.g. through the process discussed above with reference to FIG. 2) the flexible film 103 will expand outwards. This may cause the flexible film 103 to conform to an adjacent heating surface and/or to be deformed into a concave shape in which it is blown outwards. This deformation causes an increase in the volume of the reaction chamber 104 and therefore, reduces the internal pressure within the reaction chamber 104. The cartridge 100 may therefore be safely disposed of after use.

As with the cartridge shown in FIGS. 1a and 1b, the walls 112 and the film 104 of the cartridge 100 are preferably optically transmissive such that they may transmit electromagnetic radiation into and out of the reaction chamber 4 during optical analysis.

The cartridge 100 of FIGS. 3a to 3d further comprises a plurality of chambers configured to receive and store fluids—e.g. samples, reagents, solvents. Ports 113 by which these chambers (not shown) may be filled and emptied are shown in FIGS. 3a and 3d. A sample analysis system or user may dispense a sample into the reaction chamber from these ports (e.g. using a pipette). Equally, the sample may be produced within these chambers. For instance, DNA or RNA segments may be extracted and purified within these chambers using a sample analysis system and the purified may subsequently be transferred to the reaction chamber (e.g. for thermal cycling) via the inlet port 105.

From a comparison of FIGS. 1a and 3a, it will also be appreciated that the arrangement of the inlet channels 7, 107 and outlet channels 110, 110 differ between the cartridges 1, 100. The inlet channels 107 and outlet channels 110 of the cartridge 100 shown in FIG. 3a double back on themselves, having two adjacent sealing portions 107a, 110a that extend past one another. To reduce the risk of leakage, these adjacent sealing portions 107a, 110a can be welded closed in a single sealing step such that the inlet and outlet channels 107, 110 are each welded closed at two positions that are separated along the length of the respective channel 107, 110.

As will be seen, the inlet channel 107 comprises two parallel sealing portions 107a that are separated along the length of the inlet channel 107 by a 180 degree bend. The sealing portions 107a of the inlet channel 107 are laterally offset, the distance between the sealing portions 107a being approximately three to five times the width of the inlet channel 107. The two adjacent sealing portions 107a may both be welded closed in a single step by applying a heat probe to the cartridge 100 along path S₁ shown in FIG. 3d. This provides a highly effective seal since fluid (e.g. sample) is unlikely to escape from the reaction chamber past both welds.

Similarly, the outlet channel 110a comprises two parallel sealing portions 110a separated along the length of the outlet channel 110 by an intervening reservoir and a 180 degree bend. Again, the sealing portions 110a of the outlet channel are laterally offset, the distance between the sealing portions 110a being approximately three to five times the width of the outlet channel 110. Thus the adjacent sealing portions 110a may both be welded closed in a single step by applying a heat probe to the cartridge 100 along path S₂ shown in FIG. 3d. This again provides a highly effective seal since fluid (e.g. sample) is unlikely to escape from the reaction chamber 104 past both welds either after use or during the second filling step in which the reaction chamber 104 is pressurised (as discussed above in relation to step s105 in FIG. 2). The intervening reservoir between the sealing portions 110a further reduces the risk of leakage during the pressurising step since any sample from the reaction chamber 104 that does pass the first weld in the outlet channel 110 will fill the intervening reservoir before reaching the downstream second weld.

It will be appreciated that the cartridge 100 shown in FIGS. 3a to 3d is well suited for use in the methods discussed above in reference to FIG. 2.

In addition, the cartridge 100 comprises an excitation wall 112a and an emission wall 112b that form angularly offset faces of the reaction chamber 104. Specifically, the excitation and emission walls 112a, 112b are perpendicular to one another. The emission wall 112b forms an upper face of the reaction chamber 4 as shown in FIG. 1b extending parallel to the film 3, whereas the excitation wall 12a defines a side face of the reaction chamber 104.

The excitation and emission walls 112a, 112b are optically transmissive so that they may transmit electromagnetic radiation into and out of the reaction chamber 104. For instance, in preferred examples the excitation wall 112a and/or the emission all 112b may be substantially transparent to visible light, ultraviolet light and/or infrared light. The internal and external surfaces of the excitation and emission walls 112a, 112b (and the remaining walls 112 of the frame 102) may be polished so as to increase their optical transmissivity.

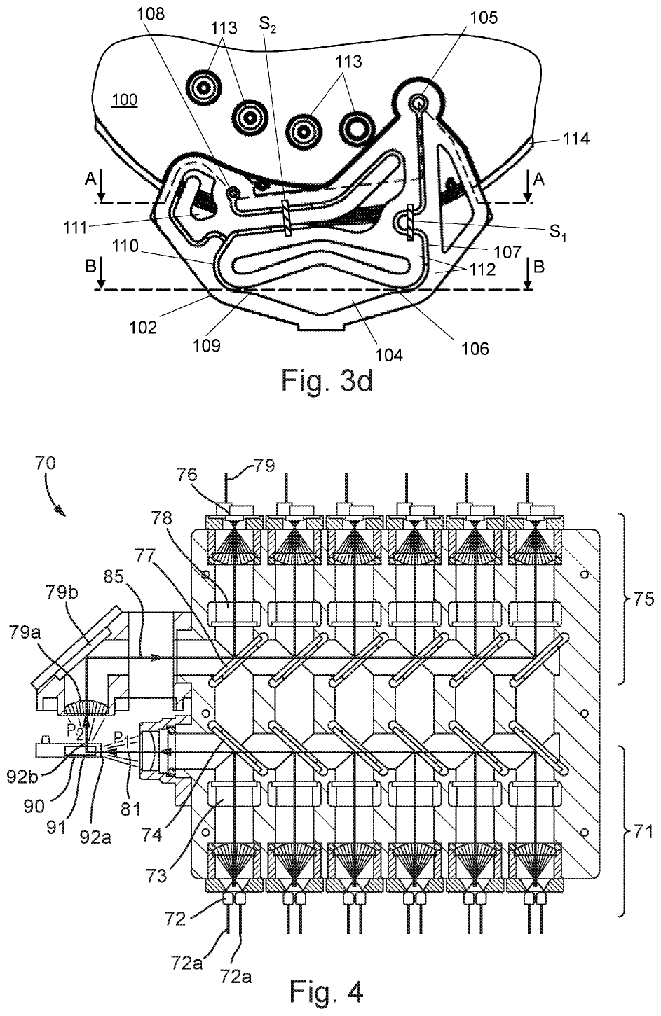

FIG. 4 schematically shows a system 70 suitable for optical analysis of the contents of a reaction chamber of a sample analysis cartridge, such as the sample analysis cartridges 1, 100 discussed above with reference to FIGS. 1 and 3.

The system 70 is arranged to analyse and interrogate the contents of a sample analysis cartridge 90 and may form part of a wider point-of-care diagnostic system. The cartridge 90 may comprise any of the features of the cartridge 10 discussed above with reference to FIGS. 1 and 3. However, this is not essential and in some cases the system 70 is configured to receive alternative cartridges. Cartridges may be inserted into the system for analysis and removed for disposal.

The system 70 comprises a source module 71 and a detection module 75. The source module 71 is configured to generate and emit electromagnetic excitation radiation 81. The source module 71 is arranged such that the excitation radiation 81 is transmitted to the reaction chamber 91 of the sample analysis cartridge 90 along a first path P₁. The detection module 75 is arranged to receive electromagnetic emission radiation 85 that is emitted from the reaction chamber 91 of the sample analysis cartridge 90 along a second path P₂. This emission radiation 85 may include light or radiation that is (for instance) reflected, transmitted, refracted or emitted from the contents of the reaction chamber 91.

As will be seen, the first path P₁ and second path P₂ are angularly offset. Specifically, as shown in FIG. 4 the first and second paths P₁, P₂ are substantially perpendicular.

The source module 71 comprises a plurality of sources 72. Specifically, it will be seen from FIG. 4 that the source module 71 comprises six sources 72. Each source 72 is configured to emit radiation having different parameters. For instance, the sources 72 may emit radiation of respective different wavelengths. The source module 71 further comprises a corresponding plurality of lenses 73 and optical filters 74 that combine the radiation from the emitters 72 into a beam of excitation radiation 81, as shown. As such, the lenses 73 and optical filters 74 form an example of a beam combining structure configured to direct excitation radiation from the plurality of sources 72 along a single path (the first path P₁) towards the sample analysis cartridge 90. Each source 72 may be operated or controlled by a controller (not shown) by connections 72a. Each sources 72 may comprise any suitable light emitter(s) including an LED and/or laser.

The detection module 75 further comprises a collimating lens 79a and a mirror 79b to direct the emission radiation 85 from the sample analysis cartridge 90 towards a plurality of detectors 76. The collimating lens 79a is configured to receive emission radiation 85 emitted across a range of angles and across a relatively wide area and form the emission radiation 85 into a beam. The beam is of higher intensity and may be more easily handled by the optics of the detection module 75.

As mentioned above, the detection module 75 further comprises a plurality of detectors 76, specifically six detectors. Each of the plurality of detectors 76 is arranged to detect radiation having a different respective parameter—e.g. radiation of different wavelengths. The detection module 75 further comprises a corresponding plurality of optical filters 77 and lenses 78 arranged to split the emission radiation 85 into a corresponding plurality of beams of radiation having these different parameters. As such, the detection module 75 is arranged such that each of these beams of radiation is made incident on a respective detector 76. As such, the optical filters 77 and lenses 78 are an example of a beam splitting structure configured to split emission radiation from the sample analysis cartridge 90 into multiple beams and to direct each beam to a respective detector 76.

The detectors 76 are configured to receive a respective beam of emission radiation 85 and to convert the emission radiation 85 into electrical signals. Subsequently, these signals may be passed to an optical analyser or other processor (not shown) for optical analysis by connections 79 such that properties of the contents of the sample analysis cartridge 90 may be determined. The detectors may comprise any suitable means for detecting light including photodetectors, photoresistors and/or photodiodes.

As discussed above, the first path P₁ of excitation radiation 81 from the source module 71 is substantially perpendicular to the second path P₂ along which the emission radiation 85 received by the detection module 75 is emitted from the cartridge 90. However, in further examples the system 70 may be arranged such that the offset between the path of the radiation emitted by the source module 71 and the path of the radiation received by the detection module 75 is in the range from 30 to 90 degrees, more preferably from 45 to 90 degrees, more preferably still from 60 to 90 degrees, more preferably still from 75 to 90 degrees. Thus interaction or interference between the emission radiation 85 and the excitation radiation 81 received by the detection module 75 is minimised. As such, the accuracy of analysis performed using the system 70 may be increased, and the time and computational resources required to isolate information in the emission radiation 85 which relates to the contents of the reaction chamber 91 from information relating the excitation radiation 81 may be reduced.

In more detail, the source module 71 is arranged to transmit the excitation radiation 81 to the reaction chamber 91 through an excitation wall 92a of the sample analysis cartridge 90. Whereas the detection module 75 is arranged to receive emission radiation 85 which was been transmitted from the reaction chamber 91 through an emission wall 92b of the sample analysis cartridge 90. The excitation wall 92a and emission wall 92b are angularly offset, being arranged as perpendicular faces of the reaction chamber 91 of the cartridge 90, and are optically transmissive such that they transmit the excitation and emission radiation 81, 85. Preferably the excitation wall 92a and emission wall 92b are colourless and substantially transparent (e.g. being formed of a suitable glass or polymer).

Preferably, the system 70 forms part of a wider point-of-care diagnostic system configured to perform PCR analysis. Such a system may additionally comprise a controller and/or optical analyser configured to control the sources 72 and to receive and analyse signals from the detectors 76.

The system may be configured to receive or accommodate a sample analysis cartridge and may further comprise a thermal surface arranged to vary the temperature of the contents of the reaction chamber, a securing mechanism configured to detachably secure the cartridge to the thermal surface such that the film is adjacent to the thermal surface, a dispensing module configured to dispense sample into an inlet port of the cartridge; and, a sealing mechanism such as a heat probe configured to seal the outlet and inlet ports of the cartridge. Thus the system is configured to perform thermocycling as occurs during PCR analysis.

The system may provide an indication to a user and/or patient of the results of analysis.

In accordance with further embodiments of the invention there are provided embodiments as set out in the following numbered clauses:
Numbered Clause 1. A point-of-care diagnostic system configured to receive a sample analysis cartridge, the system comprising:
   one or more sources configured to irradiate the reaction chamber of a sample analysis cartridge received by the diagnostic system within excitation radiation, the excitation radiation being directed towards the sample analysis cartridge along a first path; and
   one or more detectors configured to receive emission radiation from the reaction chamber, the emission radiation being emitted from the sample analysis cartridge along a second path;
   wherein the first and second paths are angularly offset.
Numbered Clause 2. A system according to numbered clause 1, wherein the angle between the first and second paths is at least 30 degrees, preferably at least 45 degrees, more preferably at least 60 degrees, more preferably still at least 75 degrees.
Numbered Clause 3. A system according to any preceding numbered clause, wherein the first and second paths are substantially perpendicular.
Numbered Clause 4. A system according to any preceding numbered clause, wherein the first path extends through an excitation wall of the reaction chamber and the second path extend through an emission wall of the reaction chamber, the excitation wall and emission wall being different.
Numbered Clause 5. A system according to any preceding numbered clause, further comprising an optical analyser configured to analyse the emission radiation received by the one or more detectors.
Numbered Clause 6. A system according to any preceding numbered clause comprising a plurality of sources each configured to emit radiation of different properties, wherein preferably said different properties comprises one or more of different wavelengths, polarisations, phases, coherences and/or amplitudes.
Numbered Clause 7. A system according to numbered clause 6, further comprising a beam combining structure configured to direct radiation emitted by the plurality of sources along the first path towards the sample analysis cartridge.
Numbered Clause 8. A system according to any preceding numbered clause, further comprising a plurality of detectors each configured to detect radiation of different properties, wherein preferably said different properties comprise one or more of different wavelengths, polarisations, phases, coherences and/or amplitude.
Numbered Clause 9. A system according to numbered clause 8, further comprising a beam splitting structure configured to split the emission radiation into a corresponding plurality of beams and to direct each beam to a respective detector of the plurality of detectors.
Numbered Clause 10. A system according to any preceding numbered clause, further comprising a collimator configured to receive emission radiation from the sample analysis cartridge and to form the emission radiation into a beam.
Numbered Clause 11. A system according to any preceding numbered clause, wherein the system further comprises a dispensing mechanism configured to introduce a sample into the reaction chamber and/or a sealing mechanism configured to seal the reaction chamber so as to prevent the escape of the contents of the reaction chamber.
Numbered Clause 12. A system according to any preceding numbered clause, wherein the system further comprises a thermal surface that is configured to heat and/or cool the contents of the reaction chamber.
Numbered Clause 13. A system according to numbered clause 12, wherein the system further comprises a securing mechanism configured to detachably retain the sample analysis cartridge against the thermal surface.
Numbered Clause 14. A system according to any preceding numbered clause further comprising a controller configured to operate the one or more sources and one or more detectors.
Numbered Clause 15. A sample analysis cartridge suitable for use in a point-of-care diagnostic system according to any of numbered clauses 1 to 14, the cartridge comprising:
   a reaction chamber configured to receive a sample;
   an excitation wall, the excitation wall forming a face of the reaction chamber and arranged to transmit excitation radiation into the reaction chamber; and,
   an emission wall, the emission wall forming a face of the reaction chamber and arranged to transmit emission radiation from the reaction chamber;
   wherein the excitation wall and emission wall are angularly offset, and wherein preferably the angle between the first and second paths is at least 30 degrees, preferably at least 45 degrees, more preferably at least 60 degrees, more preferably still at least 75 degrees.
Numbered Clause 16. A sample analysis cartridge according to numbered clause 15, wherein the emission wall and/or excitation wall is colourless and/or substantially transparent over at least a portion of the electromagnetic spectrum.
Numbered Clause 17. A sample analysis cartridge according to numbered clause 15 or numbered clause 16, wherein the area of the emission wall is greater than the area of the excitation wall, preferably at least three times greater, more preferably at least five times greater, more preferably still at least ten times greater.
Numbered Clause 18. A system comprising:
   a point-of-care diagnostic system according to any of numbered clauses 1 to 14; and
   one or more sample analysis cartridges according to any of numbered clauses 15 to 17.
Numbered Clause 19. A method performed using a point-of-care diagnostic system according to any of numbered clauses 1 to 14, the method comprising:
   inserting a sample analysis cartridge into the point-of-care diagnostic system, the sample analysis cartridge comprising a reaction chamber;
   irradiating the contents of the reaction chamber with excitation radiation from the one or more sources, the excitation radiation being directed towards the sample analysis cartridge along a first path;
   detecting emission radiation from the reaction chamber using the one or more detectors, the emission radiation being transmitted from the cartridge along a second path, wherein the first and second paths are angularly offset.
Numbered Clause 20. A method according to numbered clause 19, the method comprising:
   analysing the emission radiation to detect properties of the contents of reaction chamber.
Numbered Clause 21 A method according to either numbered clause 19 or numbered clause 20, the method further comprising:
   introducing a sample into the reaction chamber of the sample analysis cartridge, and/or
   sealing the reaction chamber of the sample analysis cartridge to prevent the contents of the reaction chamber escaping.
Numbered Clause 22. A method according to any of numbered clauses 19 to 21, the method comprising:
   securing the analysis cartridge against a thermal surface.
Numbered Clause 23. A method according to any of numbered clauses 19 to 22, wherein the sample analysis cartridge is inserted into the point-of-care diagnostic system such that the cartridge is in thermal contact with a thermal surface, and the method further comprises:
   thermal cycling the contents of the reaction chamber.

1. A sample analysis cartridge, comprising:
a reaction chamber configured to receive a sample;
a film defining at least a portion of a face of the reaction chamber;
an inlet port fluidly connected to an inlet of the reaction chamber and an outlet port fluidly connected to an outlet of the reaction chamber; and
an air reservoir fluidly connected to the reaction chamber;
the inlet port, outlet port and air reservoir arranged such that, as the reaction chamber is filled with fluid through the inlet port, gas within the reaction chamber may escape the cartridge through the outlet port whilst gas within the air reservoir is retained;
the inlet port, outlet port and air reservoir being further arranged such that such that, if the inlet and outlet ports are sealed, gas within the air reservoir applies pressure on the contents of the reaction chamber.

2. A sample analysis cartridge according to any preceding claim, further comprising a frame defining the lateral extents of the reaction chamber, wherein the film is more flexible than the frame.

3. A sample analysis cartridge according to any preceding claim, wherein the inlet and/or outlet port is permanently sealable.

4. A sample analysis cartridge according to any preceding claim, comprising:
an inlet channel extending between the inlet port and an inlet of the reaction chamber, the inlet channel comprising two adjacent sealing portions that extend past one other; and/or
an outlet channel extending between an outlet of the reaction chamber and the outlet port, the outlet channel comprising two adjacent sealing portions that extend past one another.

5. A sample analysis cartridge according to any preceding claim, wherein a lateral dimension of the reaction chamber parallel to the plane in which the film extends is at least three times the thickness of the reaction chamber perpendicular to the plane in which the film extends, preferably at least five times the thickness, more preferably ten times the thickness.

6. A sample analysis cartridge according to any preceding claim, the cartridge further comprising:
an excitation wall, the excitation wall forming a face of the reaction chamber and arranged to transmit excitation radiation into the reaction chamber; and,
an emission wall, the emission wall forming a face of the reaction chamber and arranged to transmit emission radiation from the reaction chamber;
wherein the excitation wall and emission wall are angularly offset, and wherein preferably the angle between the excitation wall and emission wall is at least 30 degrees, preferably at least 45 degrees, more preferably at least 60 degrees, more preferably still at least 75 degrees.

7. A method for preparing a sample using a sample analysis cartridge, the cartridge comprising:
a reaction chamber configured to receive a sample;
a film defining at least a portion of a face of the reaction chamber;
an inlet port fluidly connected to an inlet of the reaction chamber and an outlet port fluidly connected to an outlet of the reaction chamber; and
an air reservoir fluidly connected to the reaction chamber;
the inlet port, outlet port and air reservoir arranged such that as the reaction chamber is filled with fluid through the inlet port gas within the reaction chamber may escape the cartridge through the outlet port whilst gas within the air reservoir is retained;
the inlet port, outlet port and air reservoir being further arranged such that, if the inlet and outlet ports are sealed, gas within the air reservoir applies pressure on the contents of the reaction chamber;
wherein the method comprises:
securing the cartridge to a thermal surface such that the film is adjacent to the thermal surface, the thermal surface being arranged to vary the temperature of the contents of the reaction chamber;
a first filling step in which sample is dispensed via the inlet port so as to fill the reaction chamber, during which gas within the reaction chamber escapes the reaction chamber via the outlet port whilst gas within the air reservoir is retained within the air reservoir;
sealing the outlet port;
a second filling step in which further fluid is dispensed ivia the inlet port so as to pressurise the reaction chamber such that at least a portion of the film conforms to the thermal surface;
sealing the inlet port; and,
varying the temperature of the sample using the thermal surface.

8. A method according to claim 7, wherein said sample dispensed into the reaction chamber during first filling step comprises a purified eluate containing DNA or RNA segments.

9. A method according to claim 7 or 8, wherein securing the cartridge comprises securing the cartridge between two opposing thermal surfaces arrange to vary the temperature of the reaction chamber.

10. A method according to any of claims 7 to 9, wherein sealing the inlet port and/or the outlet port comprises permanently sealing the respective port.

11. A method according to any of claims 7 to 10 wherein sealing the outlet port comprises welding closed an outlet channel extending between an outlet of the reaction chamber and the outlet port and/or wherein sealing the inlet port comprises welding closed an inlet channel extending between the inlet port and an inlet of the reaction chamber.

12. A method according to claim 11, wherein sealing the outlet port comprises welding closed the outlet channel at two positions separated along the length of the channel and/or wherein sealing the inlet port comprises welding closed the inlet channel at two positions separated along the length of the channel.

13. A method according to any of claims 7 to 12, wherein varying the temperature of the sample using the thermal surface comprises thermal cycling the sample.

14. A method according to any of claims 7 to 13, wherein the method comprises the further step of releasing the cartridge, such that the film expands outwards, reducing the pressure within the reaction chamber.

15. A method according to any of claims 7 to 14, wherein the method further comprises optically analysing the sample using the sample analysis cartridge, wherein the method preferably comprises irradiating the sample within the reaction chamber; and receiving radiation emitted from the reaction chamber.

16. A method according to claim 15, further comprising:
irradiating the contents of the reaction chamber with excitation radiation from one or more sources, the excitation radiation being directed towards the sample analysis cartridge along a first path; and
detecting emission radiation from the reaction chamber using the one or more detectors, the emission radiation being transmitted from the cartridge along a second path, wherein the first and second paths are angularly offset.

17. A sample analysis system configured to receive a cartridge according to any of claims 1 to 6, the system comprising:
a thermal surface arranged to vary the temperature of the contents of the reaction chamber;
a securing mechanism configured to detachably secure the cartridge to the thermal surface such that the film is adjacent to the thermal surface;
a dispensing module configured to dispense sample into the inlet port of the cartridge; and,
a sealing mechanism configured to seal the outlet and inlet ports of the cartridge.

18. A sample analysis system according to claim 17, the system comprising;
one or more sources configured to irradiate the reaction chamber of a sample analysis cartridge received by the diagnostic system within excitation radiation, the excitation radiation being directed towards the sample analysis cartridge along a first path; and
one or more detectors configured to receive emission radiation from the reaction chamber, the emission radiation being emitted from the sample analysis cartridge along a second path;
wherein the first and second paths are angularly offset.

19. A sample analysis system according to claim 17 or claim 18 configured to perform the method of any of claims 7 to 16.

20. A system comprising a sample analysis system according any of claims 17 to 19 and a sample analysis cartridge according to any of claims 1 to 6.
